(19) United States
(12) Patent Application Publication
(10) Pub. No.: US 2015/0190470 A1
(43) Pub. Date: Jul. 9, 2015

(54) COMBINATION THERAPY

(71) Applicant: TETRALOGIC PHARMACEUTICALS CORPORATION, Malvern, PA (US)

(72) Inventors: C. Glenn Begley, Westlake Village, CA (US); Christopher Benetatos, Downington, PA (US); Srinivas Chunduru, West Chester, PA (US)

(73) Assignee: TETRALOGIC PHARMACEUTICALS CORPORATION, Malvem, PA (US)

(21) Appl. No.: 14/418,752

(22) PCT Filed: Aug. 1, 2013
(86) PCT No.: PCT/US2013/053126

§ 371 Date: Jan. 30, 2015

Related U.S. Application Data
(60) Provisional application No. 61/678,360, filed on Aug. 1, 2012.

(51) Int. Cl. A61K 38/19; A61N 5/06; A61K 45/06; A61N 5/10; A61K 31/409; A61K 9/00

(52) U.S. Cl. CPC A61K 38/193; A61K 31/409; A61K 9/0019; A61K 45/06; A61N 5/10; A61N 5/062

(57) ABSTRACT

A combination therapy comprising administration of a Smac mimetic and GM-CSF.

### CROSS REFERENCE TO RELATED APPLICATION

This application claims the benefit of U.S. Provisional 61/678,360, filed Aug. 1, 2012, the entire disclosure of which is incorporated herein by reference in its entirety.

### FIELD OF THE INVENTION

This invention is in the field of compositions and methods to treat proliferative disorders including cancers.

### BACKGROUND OF THE INVENTION

Inhibitors of Apoptosis Proteins (IAPs) are naturally occurring intra-cellular proteins that suppress caspase-dependent apoptosis. Smac, also known as DIABLO, is another intracellular protein that functions to antagonize, i.e., inhibit the activity of IAPs. In normal healthy cells, Smac and IAPs function together to maintain the viability of healthy cells. However, in certain disease states, e.g., cancers and other proliferative disorders, IAPs are not adequately antagonized and therefore prevent apoptosis and cause or exacerbate abnormal proliferation and survival.

Smac mimetics, also known as IAP antagonists, are synthetic small molecules that mimic the structure and IAP antagonist activity of the four N-terminal amino acids of Smac. (Smac mimetics are sometimes referred to as IAP antagonists.) When administered to animals suffering proliferative disorders, the Smac mimetics antagonize IAPs, causing an increase in apoptosis among abnormally proliferating cells. Various Smac mimetics are in development for use in the treatment of proliferative disorders.

Granulocyte macrophage colony-stimulating factor (GM-CSF) is a cytokine expressed and secreted by macrophages. A major function of GM-CSF is to aid in fighting infection by stimulating production of monocytes, which mature into macrophages, and granulocytes, i.e., neutrophils, basophils, and eosinophils. Recombinant GM-CSF made in S. cerevisiae is sold as a drug product under the brand name Leukine and the generic name sargramostim.

### SUMMARY OF THE INVENTION

Inhibitor of Apoptosis Proteins (IAPs) regulate diverse extrinsic and intrinsic cellular apoptotic signals. Among the 8 human IAPs, cIAP1, cIAP2 and XIAP have recently been identified as the primary targets of small molecule Smac-mimetic compounds. Similar to endogenous Smac, Smac mimetics bind to the conserved BIR domains of the primary IAP target proteins and antagonize their anti-apoptotic functions. Smac mimetics have been shown to enhance the cytotoxicity of chemotherapeutic drugs as well as biologic agents (TNFα and TRAIL) in vitro and in vivo. Several small molecule Smac mimetics are currently in clinical testing as cancer therapeutics. Here, we demonstrate that culture supernatants from GM-CSF-treated human peripheral blood mononuclear cells (PBMCs) sensitized the MDA-MB-231 breast cancer cell line (Smac mimetic resistant variant) to Smac mimetic-mediated apoptosis induction in a TNF dependent manner.

This invention, in one aspect, is a method of treating a proliferative disorder, such as a cancer, in a mammalian subject, e.g., a human patient, by internally administering to the subject an effective amount of a Smac mimetic and an effective amount of GM-CSF.

In a related illustrative embodiment, the invention comprises a method of sensitizing abnormally proliferating cells to apoptosis that comprises contacting the cells with a Smac mimetic and GM-CSF. Such method also can be used, e.g., to potentiate the activity of other chemotherapeutic agents, such as are described elsewhere herein.

In an illustrative embodiment, the method comprises administering to the subject an effective amount of N-{1S-[2R-(6,6′-Difluoro-3′-{4S-hydroxy-1-[2S-(2S-methylamino-propionylamino)-butyryl]-pyrrolidin-2R-ylmethyl}-1H,1′H-[2,2′]biindolyl-3-ylmethyl)-4S-hydroxy-pyrrolidine-1-carbonyl]-propyl}-2S-methylamino-propionamide (“Compound 15”) or a pharmaceutically acceptable salt thereof, in combination with GM-CSF.

Compound 15 has the following structure:

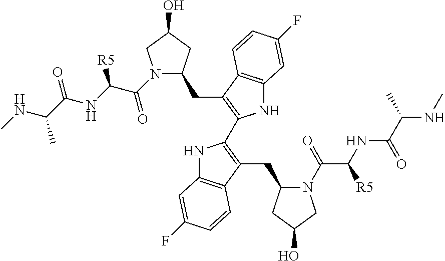

wherein R5 is —CH2CH3.

Compound 15 is also known as TL32711 and also as birinapant.

The invention, in related aspects, comprises a pharmaceutical composition comprising a Smac mimetic and GM-CSF.

In other aspects, the invention comprises a method of treating a proliferative disorder, such as a cancer, or an autoimmune disease, the symptoms of which disorder or disease can be ameliorated by pro-apoptotic therapy, in a mammalian subject in need thereof, e.g., a human, or a companion animal, a food animal, or a sporting animal, that comprises internally administering to the subject an effective amount of a Smac mimetic and an effective amount of GM-CSF.

In another illustrative embodiment, the invention comprises a method for inducing apoptosis in a cell comprising contacting the cell with a Smac mimetic and with GM-CSF. In this embodiment, the cell can be, e.g., a cancerous cell.

In additional illustrative embodiments, the invention comprises any one or more of the above methods that further comprises administering a second cancer-related therapy, such as, e.g., radiation, chemotherapy, immunotherapy, photodynamic therapy, and combinations thereof in addition to a Smac mimetic and GM-CSF.

In a further illustrative embodiment, the invention comprises a method of treating an autoimmune disease, in which the condition is caused or exacerbated by abnormal regulation of apoptosis, in a mammal in need thereof, including, for example, systemic lupus erythematosus, psoriasis, and idiopathic thrombocytopenic purpura (Morbus Werlhof) that comprises internally administering to the animal an effective amount of a Smac mimetic and of GM-CSF.

### BRIEF DESCRIPTION OF THE FIGURES

FIGS. 1(a), (b), (c), and (d) comprise data from Example 1 showing that ex-vivo treatment of PBMCs taken from Donors 1, 2, 3, and 4, respectively, with GM-CSF results in production of TNFα.

FIGS. 2(a), (b), and (c) comprise additional data from Example 1 showing that ex-vivo treatment of PBMCs taken from Donors 1, 2, 3, and 4, respectively, with GM-CSF results in production of TNFα.

FIGS. 3(a), (b), and (c) comprise data from Example 2 showing that GM-CSF-treated culture media from PBMCs taken from Donors 1, 3, and 4, respectively, sensitizes MDA-MB-231 cells to a Smac mimetic in a TNFα dependent manner.

FIG. 4 comprises data from Example 4 showing that GM-CSF+birinapant synergistically increased survival time for mice with RenCa xenografts relative to GM-CSF alone and birinapant alone.

### DETAILED DESCRIPTION OF THE INVENTION

In accordance with this invention, a Smac mimetic and GM-CSF are used in the treatment of proliferative disorders, e.g.: various benign tumors or malignant tumors (cancer), benign proliferative diseases (e.g., psoriasis, benign prostatic hypertrophy, and restenosis), or autoimmune diseases (e.g., autoimmune proliferative glomerulonephritis, lymphoproliferative autoimmune responses).

Some embodiments of the invention include inducing apoptosis of cells, particularly pathologically proliferating cells. The methods can be carried out in vitro or in vivo.

The methods of the invention can include administration of a Smac mimetic and GM-CSF, with or without one or more additional IAP antagonists, and with or without one or more additional chemotherapeutic agents. Administration of multiple agents can be simultaneous or sequential. Useful additional chemotherapeutic agents include, but are not limited to, alkylating agents (e.g., cyclophosphamide, mechlorethamine, chlorambucil, melphalan), anthracyclines (e.g., daunorubicin, doxorubicin, epirubicin, idarubicin, mitoxantrone, valrubicin), cytoskeletal disruptors (e.g., paclitaxel, docetaxel), epothilones (e.g., epothilone A, epothilone B, epothilone D), inhibitors of topoisomerase I and II (e.g., irinotecan, topotecan, etoposide, teniposide, tafluposide), nucleotide analogs precursor analogs (e.g., azacitidine, azathioprine, capecitabine, cytarabine, doxifluridine, fluorouracil, gemcitabine, mercaptopurine, methotrexate, tioguanine), peptide antibiotics (e.g., bleomycin), platinum-based agents (e.g., carboplatin, cisplatin, oxaliplatin), retinoids (e.g., all-trans retinoic acid), and vinca alkaloids and derivatives (e.g., vinblastine, vincristine, vindesine, vinorelbine). In some embodiments, the chemotherapeutic agents include fludarabine, doxorubicin, paclitaxel, docetaxel, camptothecin, etoposide, topotecan, irinotecan, cisplatin, carboplatin, oxaliplatin, amsacrine, mitoxantrone, 5-fluoro-uracil, or gemcitabine. Combination therapies can also employ such biological agents as a Type I or a Type III interferon, e.g., Interferon-α, Interferon-β and/or Interferon-δ.

Smac mimetics include, without limitation, the IAP antagonists disclosed in U.S. Pat. No. 7,517,906; U.S. Pat. No. 7,419,975; U.S. Pat. No. 7,589,118; U.S. Pat. No. 7,932,382; U.S. Pat. No. 7,345,081; U.S. Pat. No. 7,244,851; U.S. Pat. No. 7,674,787; U.S. Pat. No. 7,772,177; U.S. Pat. No. 7,989,441; U.S. Pat. No. 8,163,792; U.S. Pat. No. 8,278,293; US20100324083; US20100056467; US20090069294; US20110065726; US20110206690; WO2011098904.

The compounds disclosed therein, and Smac mimetics generally, have the structure:

[P1-P2-P3-P4] (Formula I)

or

[P1-P2-P3-P4]-L-[P1′-P2′-P3′-P4′] (Formula II)

wherein P1-P2-P3- and P1′-P2′-P3′- correspond to peptide replacements, i.e., peptidomimetics, of the N-terminal Ala-Val-Pro-tripeptide of mature Smac and P4 and P4′ correspond to amino acid replacements of the fourth N-terminal amino acid, Phe, Tyr, Ile, or Val, and L is a linking group or bond covalently linking [P1-P2-P3-P4] to [P1′-P2′-P3′-P4′].

For example, without limitation, a Smac mimetic may reside in the following genus of compounds of Formula II:

### P1 and P1′ are NHR¹—CHR²—C(O)—;

### P2 and P2′ are —NH—CHR³—C(O)—;

P3 and P3′ are pyrrolidine, pyrrolidine fused to a cycloalkyl, or pyrrolidine fused to a heterocycloalkyl having a —N— heteroatom, optionally substituted in each case, and wherein the pyrrolidine of P3/P3′ is bound to P2/P2′ by an amide bond;

### P4 and P4′ are -M-Qₚ-R⁷.

The variable substituents can be, for example:
R¹: —H or —CH3;

### R²: —CH3, —CH2CH3 or —CH2OH;

R³: C2-6 alkyl, C2-6 alkoxy, C3-C6 cycloalkyl or heterocycloalkyl, or C6-C8 aryl or heteroaryl, optionally substituted in each case;
M: a covalent bond, C1-6 alkylene, substituted C1-C6 alkylene such as but not limited to —C(O)—;
Q: a covalent bond, C1-6 alkylene, substituted C1-C6 alkylene, —O— or —NR⁸—, P: 0 or 1,
R⁷: cycloalkyl, cycloalkylaryl, alkylaryl, alkylheteroaryl, aryl or heteroaryl, optionally substituted in each case;
R⁸: —H or C1-6 alkyl.
L is a linking group or bond covalently linking [P1-P2-P3-P4] to [P1′-P2′-P3′-P4′].

“Alkyl” (monovalent) and “alkylene” (divalent) when alone or as part of another term (e.g., alkoxy) mean branched or unbranched, saturated aliphatic hydrocarbon group, having up to 12 carbon atoms unless otherwise specified. Examples of particular alkyl groups include, but are not limited to, methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The term, “lower,” when used to modify alkyl, alkenyl, etc., means 1 to 4 carbon atoms, branched or linear so that, e.g., the terms “lower alkyl”, “C₁-C₄ alkyl” and “alkyl of 1 to 4 carbon atoms” are synonymous and used interchangeably to mean methyl, ethyl, 1-propyl, isopropyl, 1-butyl, sec-butyl or t-butyl. Examples of alkylene groups include, but are not limited to, methylene, ethylene, n-propylene, n-butylene and 2-methyl-butylene.

The term substituted alkyl refers to alkyl moieties having substituents replacing one or more hydrogens on one or more (often no more than four) carbon atoms of the hydrocarbon backbone. Such substituents are independently selected from the group consisting of: a halogen (e.g., I, Br, Cl, or F, particularly fluoro(F)), hydroxy, amino, cyano, mercapto, alkoxy (such as a C₁-C₆ alkoxy, or a lower (C₁-C₄) alkoxy, e.g., methoxy or ethoxy to yield an alkoxyalkyl), aryloxy (such as phenoxy to yield an aryloxyalkyl), nitro, oxo (e.g., to form a carbonyl), carboxyl (which is actually the combination of an oxo and hydroxy substituent on a single carbon atom), carbamoyl (an aminocarbonyl such as NR₂C(O)—, which is the substitution of an oxo and an amino on a single carbon atom), cycloalkyl (e.g., a cycloalkylalkyl), aryl (resulting for example in aralkyls such as benzyl or phenylethyl), heterocyclylalkyl (e.g., heterocycloalkylalkyl), heteroaryl (e.g., heteroarylalkyl), alkylsulfonyl (including lower alkylsulfonyl such as methylsulfonyl), arylsulfonyl (such as phenylsulfonyl), and —OCF₃ (which is a halogen substituted alkoxy). The invention further contemplates that several of these alkyl substituents, including specifically alkoxy, cycloalkyl, aryl, heterocyclyalkyl and heteroaryl, are optionally further substituted as defined in connection with each of their respective definitions provided below. In addition, certain alkyl substituent moieties result from a combination of such substitutions on a single carbon atom. For example, an ester moiety, e.g., an alkoxycarbonyl such as methoxycarbonyl, or tert-butoxycarbonyl (Boc) results from such substitution. In particular, methoxycarbonyl and Boc are substituted alkyls that result from the substitution on a methyl group (—CH₃) of both an oxo (═O) and an unsubstituted alkoxy, e.g., a methoxy (CH₃—O) or a tert-butoxy ((OH₃)₃C—O—), respectively replacing the three hydrogens. Similarly, an amide moiety, e.g., an alkylaminocarbonyl, such as dimethlyaminocarbonyl or methylaminocarbonyl, is a substituted alkyl that results from the substitution on a methyl group (—CH₃) of both an oxo (═O) and a mono-unsubstitutedalkylamino or, diunsubstitutedalkylamino, e.g., dimethylamino (—N—(CH₃)₂), or methylamino (—NH—(CH₃)) replacing the three hydrogens (similarly an arylaminocarbonyl such as diphenylaminocarbonyl is a substituted alkyl that results from the substitution on a methyl group (—CH₃) of both an oxo (═O) and a mono-unsubstitutedaryl(phenyl)amino). Exemplary substituted alkyl groups further include cyanomethyl, nitromethyl, hydroxyalkyls such as hydroxymethyl, trityloxymethyl, propionyloxymethyl, aminoalkyls such as aminomethyl, carboxylalkyls such as carboxymethyl, carboxyethyl, carboxypropyl, 2,3-dichloropentyl, 3-hydroxy-5-carboxyhexyl, acetyl (e.g., an alkanoyl, where in the case of acetyl the two hydrogen atoms on the —CH₂ portion of an ethyl group are replaced by an oxo (═O)), 2-aminopropyl, pentachlorobutyl, trifluoromethyl, methoxyethyl, 3-hydroxypentyl, 4-chlorobutyl, 1,2-dimethyl-propyl, pentafluoroethyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro (n-butyl), 2-amino (iso-propyl), cycloalkylcarbonyl (e.g., cuclopropylcarbonyl) and 2-carbamoyloxyethyl. Particular substituted alkyls are substituted methyl groups. Examples of substituted methyl group include groups such as hydroxymethyl, protected hydroxymethyl (e.g., tetrahydropyranyl-oxymethyl), acetoxymethyl, carbamoyloxymethyl, trifluoromethyl, chloromethyl, carboxymethyl, carboxyl (where the three hydrogen atoms on the methyl are replaced, two of the hydrogens are replaced by an oxo (═O) and the other hydrogen is replaced by a hydroxy (—OH)), tert-butoxycarbonyl (where the three hydrogen atoms on the methyl are replaced, two of the hydrogens are replaced by an oxo (═O) and the other hydrogen is replaced by a tert-butoxy (—O—C(CH₃)₃), bromomethyl and iodomethyl. When the specification and especially the claims refer to a particular substituent for an alkyl, that substituent can potentially occupy one or more of the substitutable positions on the alkyl. For example, reciting that an alkyl has a fluoro substituent, would embrace mono-, di-, and possibly a higher degree of substitution on the alkyl moiety.

The term substituted alkylene refers to alkylene moieties having substituents replacing one or more hydrogens on one or more (often no more than four) carbon atoms of the hydrocarbon backbone where the alkylene is similarly substituted with groups as set forth above for alkyl.

Alkoxy is —O-alkyl. A substituted alkoxy is —O-substituted alkyl, where the alkoxy is similarly substituted with groups as set forth above for alkyl. One substituted alkoxy is acetoxy where two of the hydrogens in ethoxy (e.g., —O—CH₂—CH₃) are replaced by an oxo, (═O) to yield —O—C(O)—CH₃; another is an aralkoxy where one of the hydrogens in the alkoxy is replaced by an aryl, such as benzyloxy, and another is a carbamate where two of the hydrogens on methoxy (e.g., —O—CH₃) are replaced by oxo (═O) and the other hydrogen is replaced by an amino (e.g., —NH₂, —NHR or —NRR) to yield, for example, —O—C(O)—NH₂. A lower alkoxy is —O-lower alkyl.

“Alkenyl” (monovalent) and “alkenylene” (divalent) when alone or as part of another term mean an unsaturated hydrocarbon group containing at least one carbon-carbon double bond, typically 1 or 2 carbon-carbon double bonds, which may be linear or branched and which have at least 2 and up to 12 carbon atoms unless otherwise specified. Representative alkenyl groups include, by way of example, vinyl, allyl, isopropenyl, but-2-enyl, n-pent-2-enyl, and n-hex-2-enyl.

The terms substituted alkenyl and substituted alkenylene refer to alkenyl and alkenylene moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, mercapto, carboxyl, oxo, carbamoyl, cycloalkyl, aryl, heterocyclyl, heteroaryl, alkylsulfonyl, arylsulfonyl and —OCF₃.

“Alkynyl” means a monovalent unsaturated hydrocarbon group containing at least one carbon-carbon triple bond, typically 1 carbon-carbon triple bond, which may be linear or branched and which have at least 2 and up to 12 carbon atoms unless otherwise specified. Representative alkynyl groups include, by way of example, ethynyl, propargyl, and but-2-ynyl.

“Cycloalkyl” when alone or as part of another term means a saturated or partially unsaturated cyclic aliphatic hydrocarbon group (carbocycle group), having 3 to 8 carbon atoms unless otherwise specified, such as cyclopropyl, cyclobutyl, cyclopentyl and cyclohexyl, and further includes polycyclic, including fused cycloalkyls such as 1,2,3,4-tetrahydonaphthalenyls (1,2,3,4-tetrahydonaphthalen-1-yl, and 1,2,3,4-tetrahydonaphthalen-2-yl), indanyls (indan-1yl, and indan-2-yl), isoindenyls (isoinden-1-yl, isoinden-2-yl, and isoinden-3-yl) and indenyls (inden-1-yl, inden-2-yl and inden-3-yl). A lower cycloalkyl has from 3 to 6 carbon atoms and includes cyclopropyl, cyclobutyl, cyclopentyl and cyclohexyl.

The term substituted cycloalkyl refers to cycloalkyl moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), substituted alkoxy, aryloxy (such as phenoxy), nitro, mercapto, carboxyl, oxo, carbamoyl, alkyl, substituted alkyls such as trifluoromethyl, aryl, substituted aryls, heterocyclyl, heteroaryl, alkylsulfonyl, arylsulfonyl and —OCF₃.

When the specification and especially the claims refer to a particular substituent for a cycloalkyl, that substituent can potentially occupy one or more of the substitutable positions on the cycloalkyl. For example, reciting that a cycloalkyl has a fluoro substituent, would embrace mono-, di-, and a higher degree of substitution on the cycloalkyl moiety. Examples of cycloalkyls include cyclopropy, cyclobutyl, cyclopentyl, cyclohexyl, tetrahydronaphthyl and indanyl.

“Aryl” when used alone or as part of another term means an aromatic carbocyclic group whether or not fused having the number of carbon atoms designated, or if no number is designated, from 6 up to 14 carbon atoms. Particular aryl groups include phenyl, naphthyl, biphenyl, phenanthrenyl, naphthacenyl, indolyl, and the like (see e. g. Lang's Handbook of Chemistry (Dean, J. A., ed) 13^{th} ed. Table 7-2 [1985]).

The term substituted aryl refers to aryl moieties having substituents replacing one or more hydrogens on one or more (usually no more than six) carbon atoms of the aromatic hydrocarbon core. Such substituents are independently selected from the group consisting of: halo (e.g., I, Br, Cl, F), hydroxy, amino, cyano, alkoxy (such as C₁-C₆ alkoxy and particularly lower alkoxy), substituted alkoxy, aryloxy (such as phenoxy), nitro, mercapto, carboxyl, carbamoyl, alkyl, substituted alkyl (such as trifluoromethyl), aryl, —OCF₃, alkylsulfonyl (including lower alkylsulfonyl), arylsulfonyl, heterocyclyl and heteroaryl. Examples of such substituted phenyls include but are not limited to a mono- or di (halo) phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl; 3-fluorophenyl, 4-fluorophenyl, a mono- or di (hydroxy) phenyl group such as 4-hydroxyphenyl, 3-hydroxyphenyl, 2,4-dihydroxyphenyl, the protected-hydroxy derivatives thereof; a nitrophenyl group such as 3- or 4-nitrophenyl; a cyanophenyl group, for example, 4-cyanophenyl; a mono- or di (lower alkyl) phenyl group such as 4-methylphenyl, 2,4-dimethylphenyl, 2-methylphenyl, 4-(iso-propyl) phenyl, 4-ethylphenyl, 3-(n-propyl) phenyl; a mono or di (alkoxy) phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy) phenyl, 4-(t-butoxy) phenyl, 3-ethoxy-4-methoxyphenyl; 3- or 4-trifluoromethylphenyl; a mono- or dicarboxyphenyl or (protected carboxy) phenyl group such 4-carboxyphenyl; a mono- or di (hydroxymethyl) phenyl or (protected hydroxymethyl) phenyl such as 3-(protected hydroxymethyl) phenyl or 3,4-di(hydroxymethyl) phenyl; a mono- or di (aminomethyl) phenyl or (protected aminomethyl) phenyl such as 2-(aminomethyl) phenyl or 2,4-(protected aminomethyl) phenyl; or a mono- or di (N-(methylsulfonylamino)) phenyl such as 3-(N-methylsulfonylamino) phenyl. Also, the substituents, such as in a disubstituted phenyl groups, can be the same or different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, as well as for trisubstituted phenyl groups where the substituents are different, as for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups are 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. When the specification and especially the claims refer to a particular substituent for an aryl, that substituent can potentially occupy one or more of the substitutable positions on the aryl. For example, reciting that an aryl has a fluoro substituent, would embrace mono-, di-, tri, tetra and a higher degree of substitution on the aryl moiety. Fused aryl rings may also be substituted with the substituents specified herein, for example with 1, 2 or 3 substituents, in the same manner as substituted alkyl groups. The terms aryl and substituted aryl do not include moieties in which an aromatic ring is fused to a saturated or partially unsaturated aliphatic ring.

“Heterocyclic group”, “heterocyclic”, “heterocycle”, “heterocyclyl”, “heterocycloalkyl” or “heterocyclo” alone and when used as a moiety in a complex group, are used interchangeably and refer to any mono-, bi-, or tricyclic, saturated or unsaturated, non-aromatic hetero-atom-containing ring system having the number of atoms designated, or if no number is specifically designated then from 5 to about 14 atoms, where the ring atoms are carbon and at least one heteroatom and usually not more than four heteroatoms (i.e., nitrogen, sulfur or oxygen). Included in the definition are any bicyclic groups where any of the above heterocyclic rings are fused to an aromatic ring (i.e., an aryl (e.g., benzene) or a heteroaryl ring). In a particular embodiment the group incorporates 1 to 4 heteroatoms. Typically, a 5-membered ring has 0 to 1 double bonds and a 6- or 7-membered ring has 0 to 2 double bonds and the nitrogen or sulfur heteroatoms may optionally be oxidized (e. g. SO, SO₂), and any nitrogen heteroatom may optionally be quaternized. Particular unsubstituted non-aromatic heterocycles include morpholinyl (morpholino), pyrrolidinyls, oxiranyl, indolinyls, 2,3-dihydoindolyl, isoindolinyls, 2,3-dihydoisoindolyl, tetrahydroquinolinyls, tetrahydroisoquinolinyls, oxetanyl, tetrahydrofuranyls, 2,3-dihydrofuranyl, 2H-pyranyls, tetrahydropyranyls, aziridinyls, azetidinyls, 1-methyl-2-pyrrolyl, piperazinyls and piperidinyls.

The term substituted heterocyclo refers to heterocyclo moieties having substituents replacing one or more hydrogens on one or more (usually no more than six) atoms of the heterocyclo backbone. Such substituents are independently selected from the group consisting of: halo (e.g., I, Br, Cl, F), hydroxy, amino, cyano, alkoxy (such as C₁-C₆ alkoxy), substituted alkoxy, aryloxy (such as phenoxy), nitro, carboxyl, oxo, carbamoyl, alkyl, substituted alkyl (such as trifluoromethyl), —OCF₃, aryl, substituted aryl, alkylsulfonyl (including lower alkylsulfonyl), and arylsulfonyl. When the specification and especially the claims refer to a particular substituent for a heterocycloalkyl, that substituent can potentially occupy one or more of the substitutable positions on the heterocycloalkyl. For example, reciting that a heterocycloalkyl has a fluoro substituent, would embrace mono-, di-, tri, tetra and a higher degree of substitution on the heterocycloalkyl moiety.

“Heteroaryl” alone and when used as a moiety in a complex group refers to any mono-, bi-, or tricyclic aromatic ring system having the number of atoms designated, or if no number is specifically designated then at least one ring is a 5-, 6- or 7-membered ring and the total number of atoms is from 5 to about 14 and containing from one to four heteroatoms selected from the group consisting of nitrogen, oxygen, and sulfur (Lang's Handbook of Chemistry, supra). Included in the definition are any bicyclic groups where any of the above heteroaryl rings are fused to a benzene ring. The following ring systems are examples of the heteroaryl groups denoted by the term “heteroaryl”: thienyls (alternatively called thiophenyl), furyls, imidazolyls, pyrazolyls, thiazolyls, isothiazolyls, oxazolyls, isoxazolyls, triazolyls, thiadiazolyls, oxadiazolyls, tetrazolyls, thiatriazolyls, oxatriazolyls, pyridyls, pyrimidinyls (e.g., pyrimidin-2-yl), pyrazinyls, pyridazinyls, thiazinyls, oxazinyls, triazinyls, thiadiazinyls, oxadiazinyls, dithiazinyls, dioxazinyls, oxathiazinyls, tetrazinyls, thiatriazinyls, oxatriazinyls, dithiadiazinyls, imidazolinyls, dihydropyrimidyls, tetrahydropyrimidyls, tetrazolo[1,5-b]pyridazinyl and purinyls, as well as benzo-fused derivatives, for example benzoxazolyls, benzofuryls, benzothienyls, benzothiazolyls, benzothiadiazolyl, benzotriazolyls, benzoimidazolyls, isoindolyls, indazolyls, indolizinyls, indolyls, naphthyridines, pyridopyrimidines, phthalazinyls, quinolyls, isoquinolyls and quinazolinyls.

The term substituted heteroaryl refers to heteroaryl moieties (such as those identified above) having substituents replacing one or more hydrogens on one or more (usually no more than six) atoms of the heteroaryl backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, mercapto, carboxyl, carbamoyl, alkyl, substituted alkyl (such as trifluoromethyl), —OCF₃, aryl, substituted aryl, alkylsulfonyl (including lower alkylsulfonyl), and arylsulfonyl. When the specification and especially the claims refer to a particular substituent for a heteroaryl, that substituent can potentially occupy one or more of the substitutable positions on the heteroaryl. For example, reciting that a heteroaryl has a fluoro substituent, would embrace mono-, di-, tri, tetra and a higher degree of substitution on the heteroaryl moiety.

Particular “heteroaryls” (including “substituted heteroaryls”) include; 1H-pyrrolo[2,3-b]pyridine, 1,3-thiazol-2-yl, 4-(carboxymethyl)-5-methyl-1,3-thiazol-2-yl, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, I-(methylsulfonic acid)-IH-tetrazol-5-yl, 2-methyl-IH-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl, 2,5-dihydro-5-oxo-6-hydroxy-2-methyl-astriazin-3-yl, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl, 8-aminotetrazolo[1,5-b]-pyridazin-6-yl, quinol-2-yl, quinol-3-yl, quinol-4-yl, quinol-5-yl, quinol-6-yl, quinol-8-yl, 2-methyl-quinol-4-yl, 6-fluoro-quinol-4-yl, 2-methyl, 8-fluoro-quinol-4-yl, isoquinol-5-yl, isoquinol-8-yl, isoquinol-1-yl, and quinazolin-4-yl. An alternative group of “heteroaryl” includes: 5-methyl-2-phenyl-2H-pyrazol-3-yl, 4-(carboxymethyl)-5-methyl-1,3-thiazol-2-yl, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-IH-tetrazol-5-yl, I-(carboxymethyl)-1H-tetrazol-5-yl, 1-(methylsulfonic acid)-IH-tetrazol-5-yl, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl.

L is a linking group or a bond covalently linking one monomer, [P1-P2-P3-P4] to the other monomer, [P1′-P2′-P3′-P4′]. Commonly, -L- links P2 to P2′ position such as at R3 or P4 to P4′ such as at M, G, Q, or R⁷, or both P2 to P2′ and P4 to P4′. L, therefore, can be a single or double covalent bond or a contiguous chain, branched or unbranched, substituted or unsubstituted, of 1 to about 100 atoms, typically 1 to about 30 atoms, e.g., an optionally substituted alkylene, alkenylene, alkylyne, cycloalkyl, alkylcycloalkyl, alkylarylalkyl chain of 2 to 20 atoms optionally with 1-4 heteroatoms selected from —O—, —NH—, and —S—. Illustrative examples of L are a single or double covalent bond, C1-12 alkylene, substituted C1-12 alkylene, C1-12 alkenylene, substituted C1-12 alkenylene, C1-12 alkynylene, substituted C1-12 alkynylene, Xₙ-phenyl-Yₙ, or Xₙ-(phenyl)₂-Yₙ, wherein X and Y are independently C1-6 alkylene, substituted C1-6 alkylene, C1-6 alkenylene, substituted C1-6 alkenylene, C1-6 alkynylene, substituted C1-6 alkynylene, or S(O)₂.

Illustrative P3/P3′ groups include, without limitation:

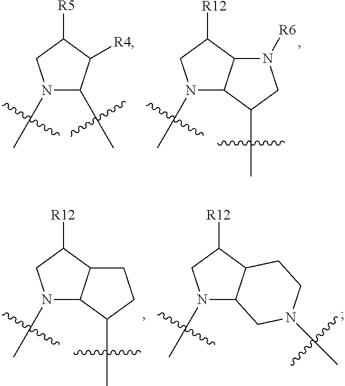

wherein
R⁶ is —H, C1-6 alkyl, substituted C1-6 alkyl, C1-6 alkoxy, substituted C1-6 alkoxy, C1-6 alkylsulfonyl, arylsulfonyl, cycloalkyl, substituted cycloalkyl, heterocycloalkyl, substituted heterocycloalkyl, aryl, substituted aryl, heteroaryl, or substituted heteroaryl;
R⁴, R⁵, and R¹² are, independently, —H, —OH, C1-6 alkyl, C1-6 heteroalkyl, C1-6 alkoxy, aryloxy, cycloalkyl, heterocycloalkyl, aryl, C1-6 alkyl aryl, or heteroaryl, or C1-6 alkyl heteroaryl, optionally substituted in each case except when R⁴ is —H or —OH.

As mentioned, in certain illustrative embodiments, the Smac mimetic used in the practice of the invention is bivalent.

Compound 15, i.e., birinapant, is an example of a specific Smac mimetic. Other illustrative examples are:

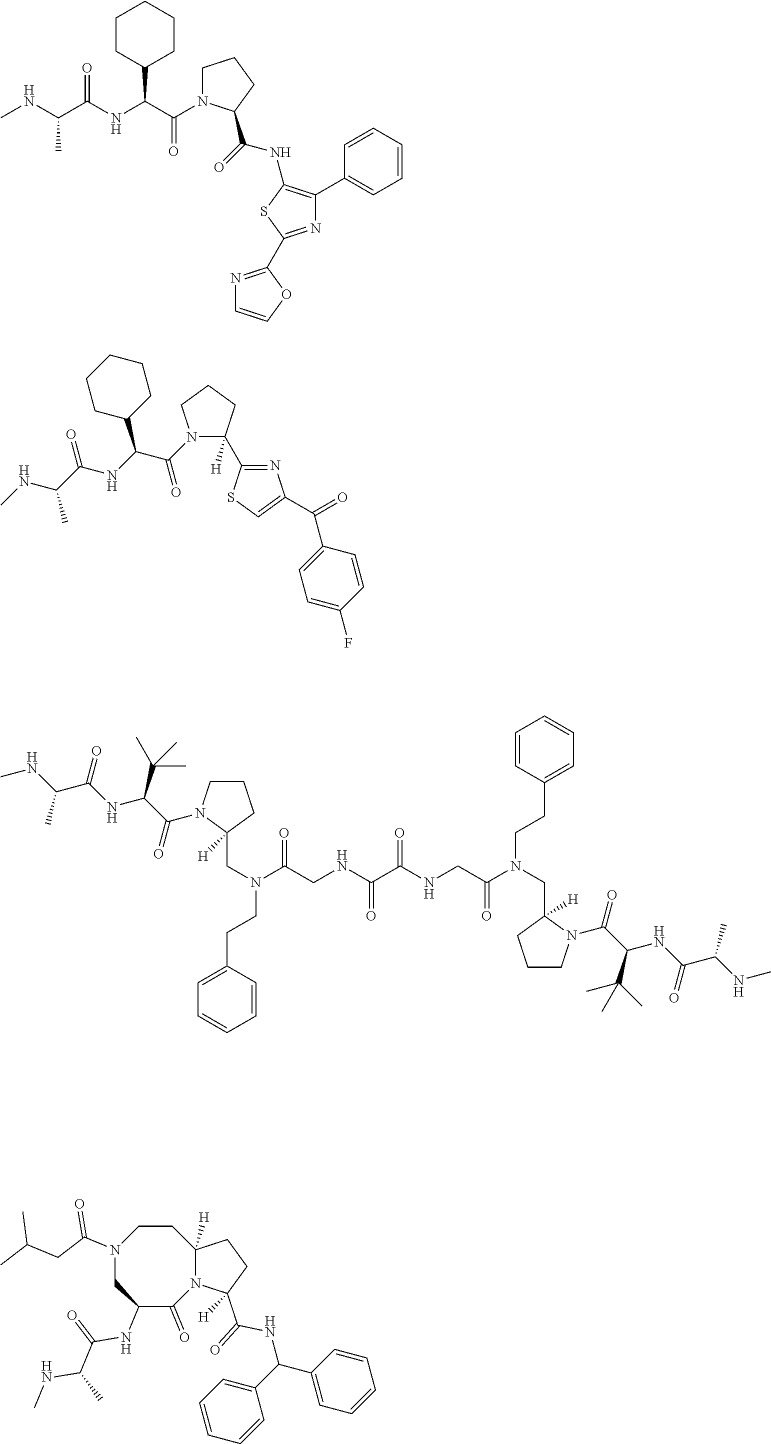

In certain illustrative embodiments, a selected Smac mimetic derepresses XIAP-mediated caspase-3 repression and/or degrades cIAP-1 not bound to TRAF2 (non TRAF2-bound, e.g., “cytoplasmic” cIAP-1 or “free” cIAP-1) as well as cIAP1 bound to TRAF2 and/or degrades cIAP-2 bound to TRAF2 but does not degrade cIAP-2 not bound to TRAF2 or weakly degrades cIAP-2 not bound to TRAF2 relative to degradation of cIAP-2 bound to TRAF2.

As used herein, the term, “GM-CSF” includes human-derived GM-CSF as well as recombinant GM-CSF. According to the prescribing information approved by the U.S. FDA, “LEUKINE is a glycoprotein of 127 amino acids characterized by three primary molecular species having molecular masses of 19,500, 16,800 and 15,500 daltons. The amino acid sequence of LEUKINE differs from the natural human GM-CSF by a substitution of leucine at position 23, and the carbohydrate moiety may be different from the native protein.” Such heterogeneity and sequence variants are of course encompassed by the term “GM-CSF” and, for the avoidance of doubt, sargramostin (e.g., LEUKINE sargramostim), is contemplated for use in the combination therapy and compositions of the invention.

In some embodiments of the invention, pharmaceutical compositions comprising a Smac mimetic and GM-CSF, alone or in combination with one or more other active pharmaceutical ingredients, are administered to a human or veterinary subject. The pharmaceutical compositions typically comprise at least one pharmaceutically acceptable excipient, e.g., a carrier or diluent, and can be administered in the conventional manner by routes including systemic, subcutaneous, topical, or oral routes. Administration may be by intravenous injection, either as a bolus or infusion, but other routes of administration, including, among others, subcutaneous or oral administration, are not precluded. An intravenous formulation can contain, e.g., from 1 mg/mL up to and including 5 mg/mL of the Smac mimetic, such as specifically Compound 15, in sterile 0.05M citrate buffered PBS, pH 5. Formulation may be by immediate release or prolonged release. Specific modes of administration and formulation will depend on the indication and other factors including the particular compound being administered. The amount of compound to be administered is that amount which is therapeutically effective, i.e., the amount that ameliorates the disease symptoms, i.e., that slows cancer progression or causes regression, without serious adverse effects relative to the disease being treated. Put another way, an effective dose is one that over the course of therapy, which may be, e.g., 1 or more weeks, e.g., multiple courses of 3 weeks on/1 week off, results in treatment of the proliferative disorder, i.e., a decrease in the rate of disease progression, termination of disease progression, or regression or remission.

The phrase “pharmaceutical composition” refers to a composition suitable for administration in medical use.

The dosage to be administered will depend on the characteristics of the subject being treated, e.g., the particular patient treated, age, weight, health, types of concurrent treatment, if any and the specific disease or disorder that is being treated. Frequency of treatments can be easily determined by one of skill in the art (e.g., by the clinician).

The dose of the Smac mimetic when given in combination with GM-CSF in accordance with this invention is expected to be the same as it would be were it administered alone, or with another, additional chemotherapeutic agent. For example, Compound 15 can be administered intravenously, e.g., by infusion, at a dose of 0.1 to 80 mg/m² of patient body surface area (BSA) per day of treatment, e.g., 2 to 80, 2 to 65, 5 to 65, 10 to 65, 20 to 65, 30 to 65, 30 or >30 to 80, 30 or >30 to 65, 30 or >30 to 60, 30 or >30 to 55, or 30 or >30 to 50 mg/m², administered, e.g., by infusion over about 1 to about 120 minutes, e.g., about 30 minutes. The dose, in most cases, will be more than 5 mg/m². For example, the dose can be in the range of 5 or >5 to 80 or 5 or >5 to 60 mg/m². Current clinical studies employ about 5 mg/m² to about 50 mg/m², specifically, 5.6 to 47 mg/m². In two patients who received 63 mg/m², weekly/3 weeks on,/1 week off, Compound 15 was not well tolerated.

It will be understood that there are different formulae for calculating BSA. Most commonly used are the Mosteller formula (Mosteller R D. “Simplified calculation of body-surface area”. N Engl J Med 317:1098 (1987)) and the Dubois & Dubois formula (Du Bois & Du Bois, Arch Intern Med 17:863 (1916)). Doses recited herein are meant to apply to BSA calculated as per any such accepted methodologies notwithstanding that such different methodologies may result in slightly different BSA calculations, e.g., depending upon the number of decimal places used. It is generally sufficient to round off BSA calculations to 1 decimal place with allowance for a reasonable margin of error, e.g., 1.6 m² (+/−0.1) or 1.9 m² (+/−0.1). For purposes of this invention, BSA can also be estimated, e.g., using relevant population averages.

Doses recited herein as mg/m² BSA can, of course, be converted to mg/kg body weight. So, for example, assuming a given patient has a BSA of 1.6 m² and a body weight of 77 kg, a dose of 40 mg/m² is equal to a dose of 64 mg, i.e., about 0.8 mg/kg. By way of further example, using an average adult BSA of 1.7 m² and an average adult body weight of 70 kg, a dose of 40 mg/m² is equal to a dose of 68 mg, i.e., also about 0.8 mg/kg. Similarly, a dose range of >30 to 60 mg/m² equates to a dose range of >0.7 mg/kg to approximately 1.5 mg/kg, in such person of average BSA and weight.

The Smac mimetic compound typically, and especially Compound 15 also has a long half-life in the patient and therefore can be administered less often than once per day. In general, Compound 15 can be administered once, twice or three times per week for one to four weeks (or longer). In some situations a treatment interval may be followed by a rest interval. A suitable rest interval includes but is not limited to one week. Such treatment cycle of one, two, three or four weeks “on” and one week “off” can be continued for as long as Compound 15 shows effectiveness and is tolerated. It should be understood that the “on” weeks are consecutive weeks, i.e., two consecutive weeks on drug, three consecutive weeks on drug, and four consecutive weeks (or more) on drug.

An illustrative dosing regimen for Compound 15 is one ˜30 minute infusion/week for one to four weeks, e.g., once a week for 2 or 3 consecutive weeks, followed by a week off. Specific illustrative dosing regimens include, without limitation, one administration by, e.g., intravenous infusion, of drug per week, in accordance with one of the following treatment cycles:

1) two weeks on/one week off, e.g., in combination with chemotherapies;
2) one week on/one week off, e.g., in patients with acute myeloid leukemia (AML);
3) two weeks on/one week off, e.g., in patients with AML;
4) three weeks on/one week off, e.g., in patients with AML;
5) continuously (i.e., without a rest interval).

An illustrative dosing regimen for Compound 15 is one 30 minute infusion/week for 2 to 4 weeks, e.g., once a week for 2 or 3 consecutive weeks, followed by a week off. Such treatment cycle of two, three or four weeks on and one week off can be continued for as long as Compound 15 shows effectiveness and is tolerated.

In an alternative dosing regimen, Compound 15 is administered weekly, twice weekly, or three times per week, without a rest interval, i.e., continuously, for as long as Compound 15 shows effectiveness and is tolerated.

When Compound 15 is used in combination therapy, the dose can be, e.g., about 5 to about 50 mg/m², or about 5 to about 40 mg/m², weekly for three weeks on/one week off or weekly continuously. An illustrative dosing regimen for Compound 15 in combination therapy is about 5 to about 35 mg/m², weekly for three weeks on/one week off or weekly continuously.

In patients in whom Compound 15 is less well tolerated, lower doses can be administered more frequently. For example, in AML patients, Compound 15 can be administered in single agent therapy at about 15 to about 20 mg/m², e.g., 17 mg/m², twice/week (e.g., Mondays and Thursdays, Tuesdays and Fridays, etc.) or 17 mg mg/m², thrice/week (e.g., Mondays, Wednesdays, Fridays) three weeks on/one week off or continuously although thrice/week dosing has not yet been studied in the clinic.

A Smac mimetic such as Compound 15 can be administered in accordance with an ascending dose protocol. An ascending dose protocol is one in which the drug is initially administered at a dose lower than the target dose and is administered at increasingly higher doses in subsequent administrations until a target dose is reached. The initial dose is a dose that is unlikely to result in an adverse event and may be sub-therapeutic. The target dose is the dose that has been determined through clinical studies to be a safe and effective dose. Dose escalation is typically carried out by increasing the dose incrementally over 3 or more administrations.

GM-CSF can also be administered intravenously although it is approved in the U.S. for administration subcutaneously and intravenously. Leukine in liquid form ready for injection contains 500 ug sargramostim at a concentration of 2.8×10⁶ IU/ml with 1.1% benzyl alcohol in a 1 ml solution. Leukine is also available in lyophilized form in vials containing 250 ug sargramostim for reconstitution with 1 ml water. The dose of the GM-CSF when given in combination with a Smac mimetic in accordance with this invention is expected to be the same as it would be were it administered alone or with another additional chemotherapeutic agent. For example, the recommended dose for GM-CSF and for Leuline sargramostim in particular is typically 250 mcg/m2/day administered intravenously. The period of time over which it is administered depends upon the particular circumstances of treatment. The recommended doses, frequency and routes of administration for GM-CSF are described in the prescribing information for Leukine sargramostim.

Thus, in general, in accordance with this invention, GM-CSF and a Smac mimetic can each be administered in accordance with a dosing regimen approved for use with each agent as monotherapy.

While it is possible to combine a Smac mimetic and GM-CSF into a single dosage unit, e.g., a sterile solution for intravenous administration, in practice, it may be preferable to administer each agent separately, e.g., using a separate pharmaceutical dosage unit, including by administering the separate dosage units according to a different dosing regimen.

Pharmaceutical compositions to be used comprise a therapeutically effective amount of the compounds (GM-CSF and Smac mimetic) as described above, or a pharmaceutically acceptable salt or other form thereof together with one or more pharmaceutically acceptable excipients. The phrase “pharmaceutical composition” refers to a composition suitable for administration in medical or veterinary use. It should be appreciated that the determinations of proper dosage forms, dosage amounts, and routes of administration for a particular patient are within the level of ordinary skill in the pharmaceutical and medical arts.

Compositions suitable for parenteral administration conveniently comprise a sterile aqueous preparation of the compounds (GM-CSF and Smac mimetic) or a composition of the invention, which is preferably isotonic with the blood of the recipient. This aqueous preparation may be formulated according to known methods using suitable dispersing or wetting agents, emulsifying and suspending agents. Various antibacterial and antifungal agents, for example, parabens, chlorobutanol, phenol, and sorbic acid also may be included. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Prolonged absorption of the injectable pharmaceutical form can be brought about by the use of agents delaying absorption, for example, aluminum monostearate and gelatin. Carrier formulation suitable for subcutaneous, intravenous, intramuscular, etc. administrations can be found in Remington's Pharmaceutical Sciences, Mack Publishing Co., Easton, Pa.

A pharmaceutical composition in intravenous unit dose form may comprise, e.g., a vial or pre-filled syringe, or an infusion bag or device, each comprising an effective amount or a convenient fraction of an effective amount such that the contents of one vial or syringe are administered at a time.

Administration can be repeated up to about 4 times per day over a period of time, if necessary to achieve a cumulative effective dose, e.g., a cumulative dose effective to produce tumor stasis or regression. A dosing regimen can be, e.g., daily or twice-weekly intravenous injections, or, e.g., once weekly injections in cycles of three weeks on and one week off for as long as the treatment is effective, e.g., until disease progresses or the drug therapy is not tolerated. The effective dose administered in each injection is an amount that is effective and tolerated.

An effective dose is one that over the course of therapy, which may be, e.g., 1 or more weeks, e.g., multiple courses of 3 weeks on/1 week off, results in treatment of the proliferative disorder, i.e., a decrease in the rate of disease progression, termination of disease progression, or regression or remission.

Solid dosage forms for oral administration include capsules, tablets, pills, powders, and granules. In such solid dosage forms, the compounds (GM-CSF and Smac mimetic) are admixed with at least one inert pharmaceutically acceptable excipient such as (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid dosage forms such as tablets, dragees, capsules, pills, and granules also can be prepared with coatings and shells, such as enteric coatings and others well known in the art. The solid dosage form also may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients. Such solid dosage forms may generally contain from 1% to 95% (w/w) of the active compounds. In certain embodiments, the active compounds generally range from 5% to 70% (w/w).

Since one aspect of the present invention contemplates the treatment of the disease/conditions with a combination of pharmaceutically active agents that may be administered separately, the invention further relates to combining separate pharmaceutical compositions in kit form. The kit comprises two separate pharmaceutical compositions: one composition contains the Smac mimetic used in the method of the present invention, and a second composition contains the GM-CSF pharmaceutical substance. The kit comprises a container for containing the separate compositions such as a divided bottle or a divided foil packet. Additional examples of containers include syringes, e.g., pre-filled syringes, boxes and bags. Typically, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing physician or veterinarian.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . . Second Week, Monday, Tuesday, . . . ” etc. Other variations of memory aids will be readily apparent. A “daily dose” can be a single tablet or capsule or several pills or capsules to be taken on a given day. Also, a daily dose of a substance of the present invention can consist of one tablet or capsule, while a daily dose of the second substance can consist of several tablets or capsules and vice versa. The memory aid should reflect this variety and aid in correct administration of the active agents.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another aspect, the invention comprises a device for intravenous infusion comprising a Smac mimetic and GM-CSF in a pharmaceutically acceptable carrier. Such device can be, e.g., a dual compartment vial having an integrated connector for joining the compartments, simultaneously or sequentially, with an intravenous tube or with a needle for intravenous injection or infusion.

Liquid dosage forms for oral administration include pharmaceutically acceptable emulsions, solutions, suspensions, syrups, and elixirs. In addition to the compounds or composition, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances. Besides such inert diluents, the composition can also include adjuvants, such as wetting agents, emulsifying and suspending agents, sweetening, flavoring, and perfuming agents.

The compounds and compositions used in the method of the present invention also may benefit from a variety of delivery systems, including time-released, delayed release or sustained release delivery systems. Such option may be particularly beneficial when the compounds and composition are used in conjunction with other treatment protocols as described in more detail below.

Many types of controlled release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, as used herein, means that the implant is constructed and arranged to deliver therapeutic levels of the active compounds for at least 30 days, and preferably 60 days. Long-term sustained release implants are well-known to those of ordinary skill in the art and include some of the release systems described above.

The compounds used in the method of the present invention and pharmaceutical compositions comprising compounds used in the method of the present invention can be administered to a subject suffering from cancer, an autoimmune disease or another disorder where a defect in apoptosis is implicated. In connection with such treatments, the patient can be treated prophylactically, acutely, or chronically using the compounds and compositions used in connection with the method of the present invention, depending on the nature of the disease. Typically, the host or subject in each of these methods is human, although other mammals may also benefit from the present invention.

As described in U.S. Pat. No. 7,244,851, IAP antagonists can be used for the treatment of all cancer types which fail to undergo apoptosis. Thus, compounds used on the method of the present invention can be used to provide a therapeutic approach to the treatment of many kinds of solid tumors, including but not limited to carcinomas, sarcomas including Kaposi's sarcoma, erythroblastoma, glioblastoma, meningioma, astrocytoma, melanoma and myoblastoma. Treatment or prevention of non-solid tumor cancers such as leukemia is also contemplated by this invention. Indications may include, but are not limited to brain cancers, skin cancers, bladder cancers, ovarian cancers, breast cancers, gastric cancers, pancreatic cancers, colon cancers, blood cancers, lung cancers and bone cancers. Examples of such cancer types include neuroblastoma, intestine carcinoma such as rectum carcinoma, colon carcinoma, familial adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basal cell carcinoma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

The inventors believe that the IAP antagonists suitable for use in the method of the present invention will be active for treating human malignancies including, but not limited to, such human malignancies in which cIAP1 and cIAP2 are over-expressed (e.g., lung cancers, see Dai et al, Hu. Molec. Genetics, 2003 v 12 pp 791-801; leukemias (multiple references), and other cancers (Tamm et al, Clin Cancer Res, 2000, v 6, 1796-1803). The inventors also expect that the IAP antagonists suitable for use in the method of the present invention will be active in disorders that may be driven by inflammatory cytokines such as TNFα playing a pro-survival role (for example, there is a well defined role for TNFα acting as a survival factor in ovarian carcinoma, similarly for gastric cancers (see Kulbe, et al, Cancer Res 2007, 67, 585-592).

In addition to apoptosis defects found in tumors, defects in the ability to eliminate self-reactive cells of the immune system due to apoptosis resistance are considered to play a key role in the pathogenesis of autoimmune diseases. Autoimmune diseases are characterized in that the cells of the immune system produce antibodies against its own organs and molecules or directly attack tissues resulting in the destruction of the latter. A failure of those self-reactive cells to undergo apoptosis leads to the manifestation of the disease. Defects in apoptosis regulation have been identified in autoimmune diseases such as systemic lupus erythematosus or rheumatoid arthritis.

Examples of such autoimmune diseases include collagen diseases such as rheumatoid arthritis, systemic lupus erythematosus, Sharp's syndrome, CREST syndrome (calcinosis, Raynaud's syndrome, esophageal dysmotility, telangiectasia), dermatomyositis, vasculitis (Morbus Wegener's) and Sjögren's syndrome, renal diseases such as Goodpasture's syndrome, rapidly-progressing glomerulonephritis and membrano-proliferative glomerulonephritis type II, endocrine diseases such as type-I diabetes, autoimmune polyendocrinopathy-candidiasis-ectodermal dystrophy (APECED), autoimmune parathyroidism, pernicious anemia, gonad insufficiency, idiopathic Morbus Addison's, hyperthyroidosis, Hashimoto's thyroiditis and primary myxedema, skin diseases such as pemphigus vulgaris, bullous pemphigoid, herpes gestationis, epidermolysis bullosa and erythema multiforme major, liver diseases such as primary biliary cirrhosis, autoimmune cholangitis, autoimmune hepatitis type-1, autoimmune hepatitis type-2, primary sclerosing cholangitis, neuronal diseases such as multiple sclerosis, myasthenia gravis, myasthenic Lambert-Eaton syndrome, acquired neuromyotony, Guillain-Barré syndrome (Müller-Fischer syndrome), stiff-man syndrome, cerebellar degeneration, ataxia, opsoklonus, sensoric neuropathy and achalasia, blood diseases such as autoimmune hemolytic anemia, idiopathic thrombocytopenic purpura (Morbus Werlhof), infectious diseases with associated autoimmune reactions such as AIDS, Malaria and Chagas disease.

The present invention can be carried out in conjunction with other treatment approaches, e.g., in combination with a biologic or chemotherapeutic agent or with chemoradiation. As discussed above, embodiments of the invention also include a method of treating a patient afflicted with cancer by the contemporaneous or concurrent administration of a biological or chemotherapeutic agent additional to the Smac mimetic, such as Compound 15. Such biological or chemotherapeutic agents include but are not limited to the chemotherapeutic agents described in “Modern Pharmacology with Clinical Applications”, Sixth Edition, Craig & Stitzel, Chpt. 56, pg 639-656 (2004). The chemotherapeutic agent can be, but is not limited to, alkylating agents, antimetabolites, anti-tumor antibiotics, plant-derived products such as taxanes, enzymes, hormonal agents, miscellaneous agents such as cisplatin, monoclonal antibodies, glucocorticoids, mitotic inhibitors, topoisomerase I inhibitors, topoisomerase II inhibitors, immunomodulating agents such as interferons, cellular growth factors, cytokines, and nonsteroidal anti-inflammatory compounds (NSAID), cellular growth factors and kinase inhibitors. Other suitable classifications for chemotherapeutic agents include mitotic inhibitors, and anti-estrogenic agents.

Specific examples of suitable biological and chemotherapeutic agents include, but are not limited to, carboplatin, cisplatin, carmustine (BCNU), 5-fluorouracil (5-FU), cytarabine (Ara-C), gemcitabine, methotrexate, daunorubicin, doxorubicin, dexamethasone, irinotecan, topotecan, etoposide, paclitaxel, docetaxel, vincristine, tamoxifen, TNFα, TRAIL and other members, i.e., other than TRAIL and TNFα, of the TNF superfamily of molecules, interferon (in both its alpha and beta forms), thalidomide, thalidomide derivatives such as lenalidomide, melphalan, and PARP inhibitors. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim and sargramostim.

Non-steroidal anti-inflammatory drugs (NSAIDs) have been shown to induce apoptosis in colorectal cells. NSAIDs appear to induce apoptosis via the release of SMAC from the mitochondria (PNAS, Nov. 30, 2004, vol. 101:16897-16902). Therefore, the use of NSAIDs in combination with the compounds and compositions that are used in the method of the present invention may increase the activity of each drug over the activity of either drug independently.

The present invention can be carried out with co-administration of TRAIL or other chemical or biological agents which bind to and activate the TRAIL receptor(s). TRAIL has received considerable attention recently because of the finding that many cancer cell types are sensitive to TRAIL-induced apoptosis, while most normal cells appear to be resistant to this action of TRAIL. TRAIL-resistant cells may arise by a variety of different mechanisms including loss of the receptor, presence of decoy receptors, or overexpression of FLIP which competes for zymogen caspase-8 binding during DISC formation. In TRAIL resistance, the compounds or compositions that are used in the method of the present invention may increase tumor cell sensitivity to TRAIL leading to enhanced cell death, the clinical correlations of which are expected to be increased apoptotic activity in TRAIL resistant tumors, improved clinical response, increased response duration, and ultimately, enhanced patient survival rate. In support of this, reduction in XIAP levels by in vitro antisense treatment has been shown to cause sensitization of resistant melanoma cells and renal carcinoma cells to TRAIL (Chawla-Sarkar, et al., 2004). The Smac mimetic compounds used in the method of the present invention bind to IAPs and inhibit their interaction with caspases, therein potentiating TRAIL-induced apoptosis.

The combination of agents used in the practice of this invention can also be applied locally, such as in isolated limb perfusion. The compounds used in the method of the invention can also be applied topically, e.g., as a cream, gel, lotion, or ointment, or in a reservoir or matrix-type patch, or in an active transdermal delivery system.

### EXAMPLES

The examples that follow were carried out with the Compound 15, which was prepared substantially as described in published U.S application US20110003877, which has issued as a patent as U.S. Pat. No. 8,283,372.

### Example 1

### Ex-Vivo Treatment of PBMCs with GM-CSF Results in Production of TNFα

### Isolation and Culture of Peripheral Blood Mononuclear Cells (PBMCs):

Peripheral blood mononuclear cells (PBMC) were isolated from two healthy male donors using BD Vacutainer® CPT cell preparation tubes (Becton, Dickinson and Company, Franklin Lakes N.J. 07417, REF 362753) according to manufacturer's protocol. After isolation, mononuclear cell layer was removed from CPT tube and transferred to a sterile 15 mL centrifuge tube. Cells were washed by adding 10 mL of Dulbecco's Modified Eagle Medium (DMEM) containing 10% fetal bovine serum (FBS) to cells, tube was inverted several times to mix and cells were pelleted by centrifugation at 1500 RPM for 10 minutes. Following centrifugation, media was removed and cells were resuspended in DMEM containing 10% FBS to a concentration of 1×10⁶ cells/mL. Cells were next seeded into 6-well dishes at 1×10⁶ cells/mL in 2 mL/well. Following 2 hr incubation at 37° C./5% CO₂, human recombinant GM-CSF (R&D Systems, Minneapolis Minn. 55413, Catalog # #215-GM) was added to cells at indicated concentrations. Following overnight incubation at 37° C./5% CO₂, media was collected into sterile 15 mL centrifuge tubes and cells were removed by centrifugation at 1500 RPM for 10 minutes. Media was transferred to new, sterile tubes and frozen at −80° C. until use.

### Determination of TNFα Concentration in Culture Supernatants of GM-CSF-Treated PBMC:

TNFα concentration in culture supernatants was determined by ELISA (BD OptEIA TNF kit II) according to manufacturer's recommendations. Absorbance values were analyzed using GraphPad Prism linear regression analysis and reported as pg/mL TNFα.

### Results

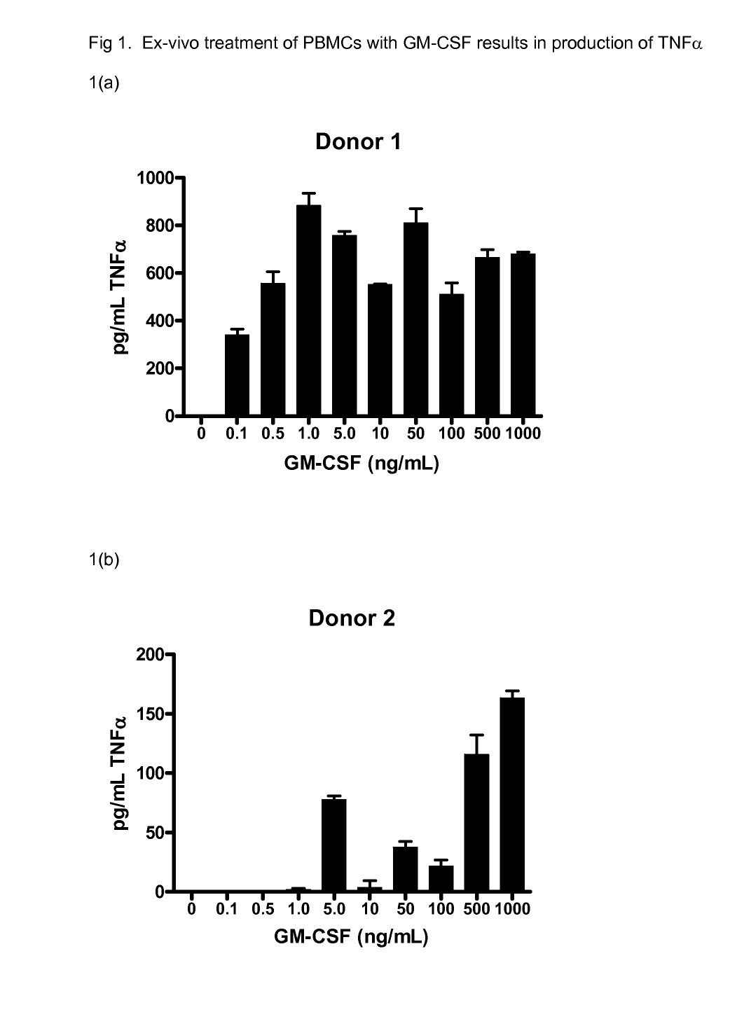

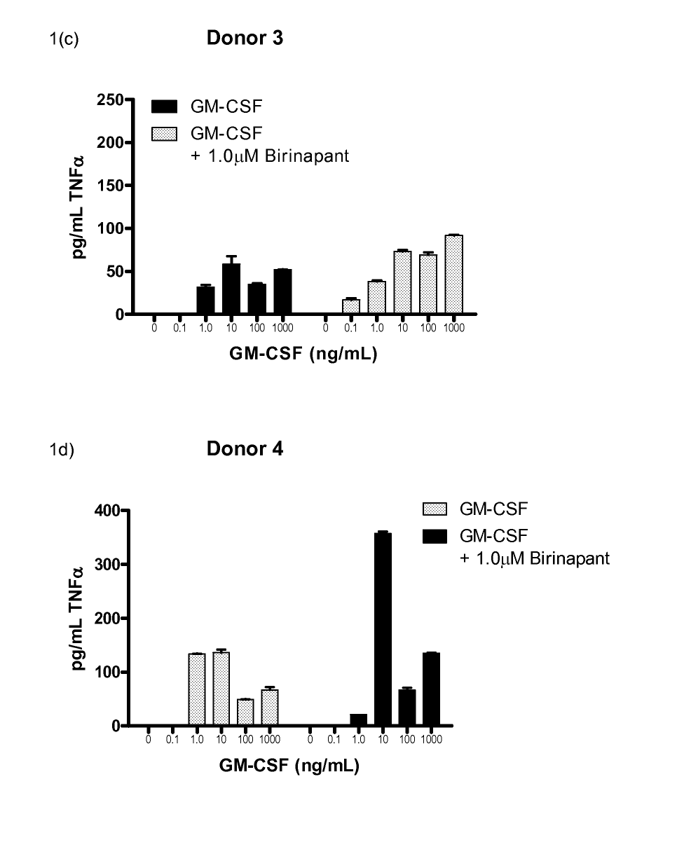

Data obtained from this experiment are illustrated in FIGS. 1(a) and (b) and demonstrate that treatment of PBMCs with GM-CSF causes increased expression and secretion of TNFα.

### Extension Studies:

In a first extension of the preceding study, substantially the same experiment was carried out with PBMCs from two additional donors, Donors 3 and 4. In this first extension study, cells were treated with GM-CSF, GM-CSF+1.0 uM birinapant. Data from this additional experiment are illustrated in FIGS. 1(c) and (d) and further demonstrate that treatment of PBMCs with GM-CSF causes increased expression and secretion of TNFα.

In this first extension study, TRAIL was undetectable by ELISA.

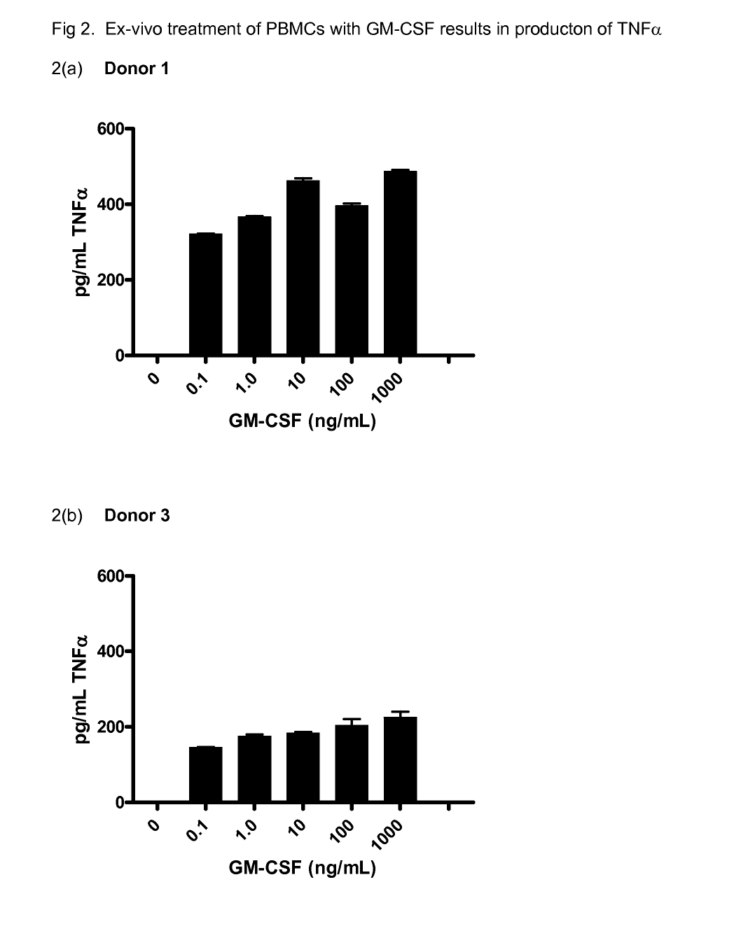

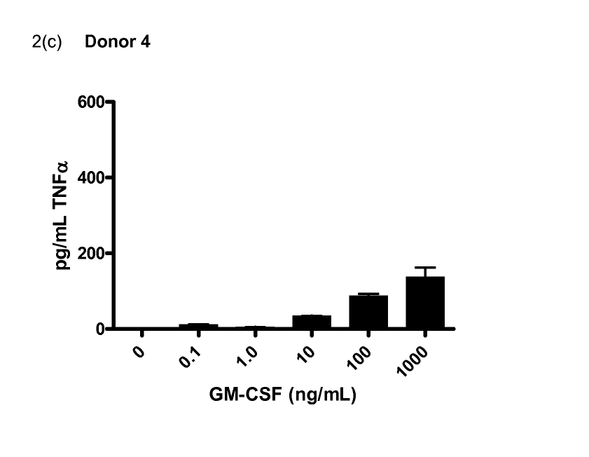

In a second extension study, substantially the same experiment was subsequently carried out with new PBMC culture supernatants from Donors 1, 3, and 4. Data from this additional experiment are illustrated in FIGS. 2(a), (b), and (c) and further demonstrate that that treatment of PBMCs with GM-CSF causes increased expression and secretion of TNFα.

### Example 2

### GM-CSF Treated Culture Media Sensitizes MDA-MB-231 Cells to Compound-15 in a TNFα Dependent Manner

MDA-MB-231 cells (human breast cancer) were seeded into 96-well plates at a density of 10,000 cells/well and allowed to adhere overnight. Next day, culture supernatants from untreated Donor 1 PBMC culture or 1 ng/mL GM-CSF-treated Donor 1 PBMC culture was added to MDA-MB-231 cells in the presence or absence of 1 μM Compound 15, 1 μM Compound 15 plus TNFα neutralizing antibody (R&D Systems #MAB610, 10 μg/mL), 1 μM Compound 15 plus TRAIL neutralizing antibodies (R&D Systems #374-DR and 631-T2, 100 ng/ml each) or 1 μM Compound 15 plus both TNFα and TRAIL neutralizing antibodies. All antibodies were tested in the absence of Compound 15 as control. Viability of MDA-MB-231 cells was measured following 24 hr incubation at 37° C./5% CO₂ by MTT (3-(4,5-dimethylthiazol-2-yl)-2,5-diphenyltetrazolium bromide) assay substantially as described in Hansen, M. B., Nielsen, S. E., and Berg, K. (1989) J. Immunol. Methods 119, 203-210.

### Results

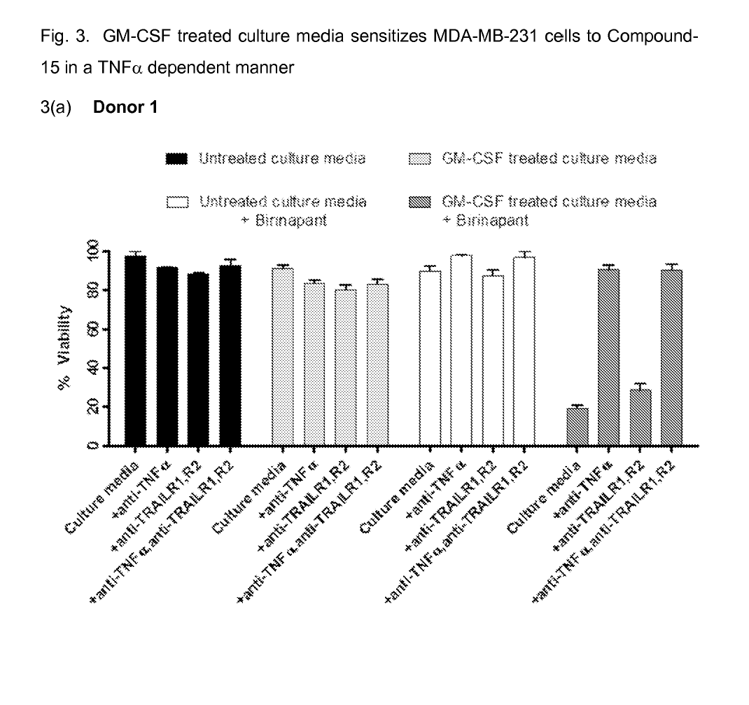

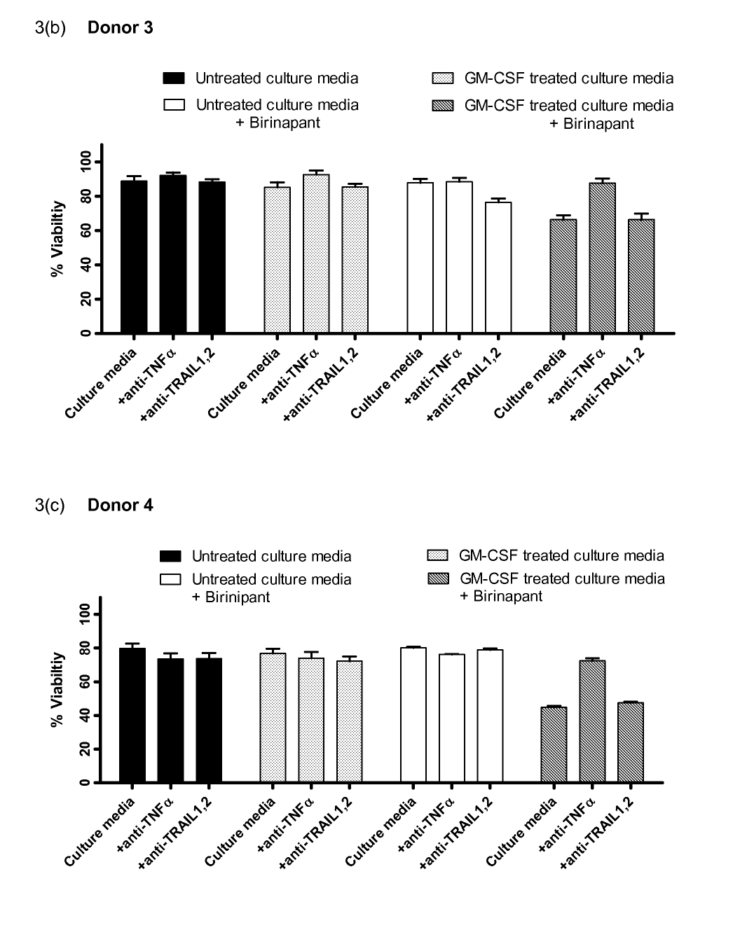

Data obtained from this experiment are illustrated in FIG. 3(a). The data show sensitization of MDA-MB-231 Smac mimetic resistant variant to Compound 15—by the supernatant obtained from the cell cultures of Example 1, resulting in induction of Smac mimetic-induced apoptosis. The synergy in all cases was blocked by TNFα antibody. These results indicate that the mechanism of synergistic induction of apoptotic death in this tumor line was through production of TNFα by the PBMCs in response to GM-CSF treatment.

### Extension Study:

In an extension study, substantially the same experiment was carried out with culture supernatants from two additional donors, Donors 3 and 4, excluding treatment with the combination of anti-TNFα and anti-TRAIL antibodies. Data from this additional experiment are illustrated in FIGS. 3(b) and (c) and further demonstrate that the mechanism of synergistic induction of apoptotic death in this tumor line was through production of TNFα by the PBMCs in response to GM-CSF treatment.

### Example 3

### TNFα, but not GM-CSF Alone, Sensitizes Birinapant Resistant Cells

Mouse renal carcinoma (RenCa) cells were seeded into 96-well plates at a density of 10,000 cells/well and allowed to adhere overnight. On the next day, birinapant alone or in combination with varying concentrations of TNFα (R&D systems) (0.000001 to 100 ng/mL) or mouse GM-CSF (R&D Systems) (0.001 to 10 ng/mL) were added. Following a 24 h incubation, viability was assessed by MTT assay.

### Results

RenCa cell viability decreased in direct relation to the concentration of TNFα, but was unaffected by addition of GM-CSF, lending further support to the conclusion that GM-CSF sensitization is TNFα dependent.

### Example 4

### GM-CSF+Birinapant Synergistically Increase Survival

40 female BALB/c mice (10 per treatment group) were inoculated with 1×10⁵ RenCa cells on each flank. The mice were subsequently treated qdX5 each week for 4 consecutive weeks with birinapant, mGM-CSF, birinapant and mGM-CSF, or a vehicle control. Each dose of birinapant was 15 mg/Kg IP and each dose of mGM-CSF was 10 mg IP.

### Results

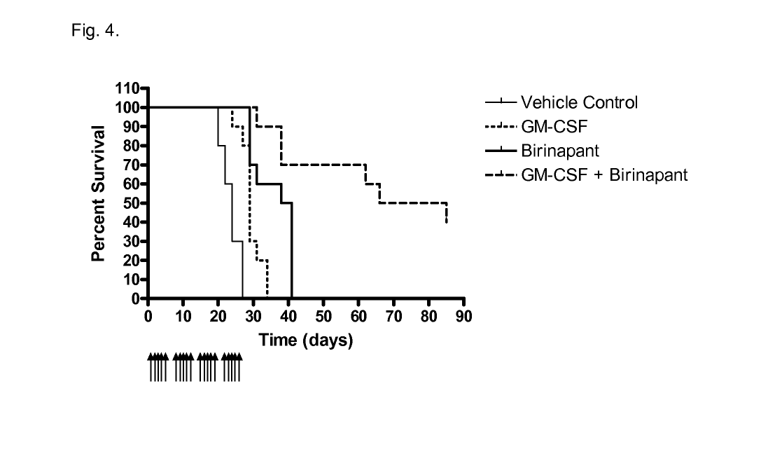

Results are shown in FIG. 4. Arrows indicate birinapant and mGM-CSF dosing. GM-CSF+birinapant synergistically increased survival time relative to GM-CSF and birinapant alone. 50% of mice survived approximately 28 days on control; approximately 30 days on mGM-CSF; approximately 40 days on birinapant, and approximately 85 days on birinapant+mGM-CSF.

Taken together, the data from Examples 1-4 indicate that a combination therapy employing both a Smac mimetic and GM-CSF provides a useful approach to inducing abnormally proliferating cells to die via apoptosis or to sensitizing such cells to die via apoptosis when also contacted with a further apoptosis-inducing agent. Thus, these data indicate that such combination therapy can be useful in the treatment of solid and hematologic malignancies.

It is understood that the examples and embodiments described herein are for illustrative purposes only and that various modifications or changes in light thereof will be suggested to persons skilled in the art and are to be included within the spirit and purview of this application and the scope of the appended claims. All patent and literature references cited herein are incorporated by reference herein as though fully set forth.

1. A method of treating a proliferative disorder in a mammal in need thereof that comprises internally administering to the animal (i) an effective amount of a Smac mimetic and (ii) an effective amount of GM-CSF.

2. The method of claim 1 wherein the proliferative disorder is a cancer.

3. The method of claim 2 wherein the proliferative disorder is a cancer selected from the group consisting of: sarcomas, bladder cancer, ovarian cancer, breast cancer, brain cancer, pancreatic cancer, colon cancer, blood cancer, skin cancer, lung cancer, and bone cancer.

4. The method of claim 2 wherein the cancer is selected from colorectal cancer, renal carcinoma, ovarian carcinoma, pancreatic carcinoma, prostate carcinoma, breast carcinoma, melanoma, glioblastoma, acute myeloid leukemia, small cell lung cell carcinoma, non-small cell lung carcinoma, rhabdomyosarcoma, and basal cell carcinoma.

5. The method of claim 2 wherein the cancer is selected from breast cancer or renal carcinoma.

6. The method of any of claims 1, 2, 3, 4, and 5 wherein the GM-CSF is recombinant GM-CSF.

7. The method of any of claims 1, 2, 3, 4, and 5 wherein the GM-CSF is sargramostim.

8. The method of any of the preceding claims wherein the Smac mimetic is Compound 15 or a pharmaceutically acceptable salt thereof.

9. The method of any of the preceding claims wherein the GM-CSF and the Smac mimetic are co-administered separately.

10. The method of claim 9 wherein the GM-CSF and the Smac mimetic are co-administered by internal administration of different pharmaceutical dosage units and at different times.

11. A method for inducing apoptosis in a cell comprising contacting the cell with a Smac mimetic and GM-CSF.

12. The method of claim 11 wherein the cell is a cancer cell.

13. The method of any one or more of the preceding claims that further comprises administering the Smac mimetic and the GM-CSF in combination with a further cancer therapy selected from radiation, chemotherapy, immunotherapy, photodynamic therapy, and combinations thereof.

14. A method of treating an autoimmune disease, in a mammal in need thereof, wherein the autoimmune disease is one in which the condition is caused or exacerbated by abnormal regulation of apoptosis and is selected from the group consisting of: systemic lupus erythematosus, psoriasis, and idiopathic thrombocytopenic purpura (Morbus Werlhof) that comprises internally administering to the animal an effective amount of a Smac mimetic and an effective amount of GM-CSF.

15. A method of sensitizing abnormally proliferating cells to apoptosis that comprises contacting the cell with a Smac mimetic and GM-CSF.

16. A pharmaceutical composition comprising a Smac mimetic and GM-CSF in a pharmaceutically acceptable carrier.

17. A device for intravenous infusion comprising a Smac mimetic and GM-CSF in a pharmaceutically acceptable carrier.

18. A Smac mimetic for co-administration with GM-CSF to a patient suffering a proliferative disorder.
